(19) United States
(12) Patent Application Publication
(10) Pub. No.: US 2026/0225987 A1
(43) Pub. Date: Aug. 6, 2026

(54) PROCESSES FOR MAKING 1,2-DIACETOXYETHANE FROM MONOETHYLENE GLYCOL BY REACTIVE DISTILLATION AND INTEGRATED VINYL ACETATE MONOMER PRODUCTION

(71) Applicant: New Product Innovation, LLC, Johnston, IA (US)

(72) Inventors: David James Schreck, Lake City, MN (US); Donald Bunning, South Charleston, WV (US); Glenn Graham, Harrison, ID (US); Louis Kapicak, Cross Lanes, WV (US)

(73) Assignee: New Product Innovation, LLC, Johnston, IA (US)

(21) Appl. No.: 19/399,297

(22) Filed: Nov. 24, 2025

Related U.S. Application Data
(60) Provisional application No. 63/723,863, filed on Nov. 22, 2024.

(51) Int. Cl. C07C 67/02; C07C 67/54

(52) U.S. Cl. CPC C07C 67/02; C07C 67/54

(57) ABSTRACT

This disclosure provides reactive distillation processes to produce ethylene glycol diacetate from monoethylene glycol and acetic acid, followed by acetic anhydride treatment to reduce 2-hydroxyethyl acetate and water to very low levels. Integration with vinyl acetate monomer cracking recovers acetic anhydride for reuse, enabling improved product purity, reduced heavies formation, lower corrosion, and meaningful capital and energy savings.

### CROSS-REFERENCES & RELATED APPLICATIONS

This application claims priority to U.S. Provisional Application No. 63/723,863 filed Nov. 22, 2024, and entitled “PROCESSES FOR MAKING 1,2-DIACETOXYETHANE FROM MONOETHYLENE GLYCOL BY REACTIVE DISTILLATION AND INTEGRATED VINYL ACETATE MONOMER PRODUCTION,” which is hereby incorporated by reference in its entirety under 35 U.S.C. § 119(e).

### TECHNICAL FIELD

This disclosure pertains to processes for converting monoethylene glycol to 1,2-diacetoxyethane (ethylene glycol diacetate) by reactive distillation, and particularly to such reactive distillation processes having enhanced economics for the production of high-quality ethylene glycol diacetate, and in a preferred aspect, to such processes integrated with vinyl acetate monomer production.

### BACKGROUND

Ethylene glycol diacetate has various uses, such as a solvent and ingredient in chemical formulations and as an intermediate, e.g., for making vinyl acetate. Vinyl acetate monomer (VAM) is an important commodity chemical with an annual global production volume in 2020 of nearly 9 million metric tonnes. An interest exists in using sustainable feedstocks rather than fossil-based feedstocks to make commodity chemicals, including VAM. Commercial acceptance of commodity chemicals made using sustainable feedstocks will, in part, depend on the cost of their production.

Numerous processes exist for making VAM. Vinyl acetate is typically made by the catalytic reaction of ethylene, acetic acid, and oxygen. Some industrial production uses the hydroesterification of acetic acid and acetylene. Ethylene is typically made from fossil fuels, although ethanol, made from sustainable resources, can be converted to ethylene for use in the typical catalytic process. Another proposed route is the thermal or catalytic cracking of 1,1-diacetoxyethane. See, for instance, U.S. Pat. No. 2,425,389 and European Patent Application Publication No. 0 048 173 A1. The high costs of producing 1,1-diacetoxyethane have not led to this proposed route achieving commercial viability for making VAM. U.S. Pat. No. 3,787,485 discloses the thermal cracking of ethylene glycol diacetate to vinyl acetate at 435° C. to 560° C. This process has also not seen commercial viability.

Nevertheless, the use of ethylene glycol diacetate to make VAM continues to be of interest as ethylene glycol diacetate can be made from monoethylene glycol, which, in turn, can be made from sustainable resources. The conventional process for making monoethylene glycol is by cracking fossil-based feedstock to produce ethylene, partially oxidizing the ethylene to produce ethylene oxide, and then hydrolyzing ethylene oxide to monoethylene glycol. The conventional process could use ethylene derived from sustainable feedstocks, e.g., ethylene made by the dehydration of ethanol derived from the fermentation of carbohydrates. Other processes exist for making monoethylene glycol from sustainable feedstocks. For instance, sustainable feedstock can be converted to syngas and monoethylene glycol can be made by the glycol oxalate process such as disclosed in U.S. Pat. No. 4,453,026. Also, recent efforts have been devoted to converting carbohydrates to monoethylene glycol by retro aldol conversion of the carbohydrate to, among others, glycol aldehyde, and hydrogenation to monoethylene glycol. See, for instance, U.S. Pat. No. 9,783,472.

In the esterification of monoethylene glycol with acetic acid, first, the half ester, 2-hydroxyethyl acetate, is produced with water being the coproduct. Then the half ester is reacted with acetic acid to make ethylene glycol diacetate and water. Each of these reactions is an equilibrium reaction, and to drive the reactions toward completion, water is removed from the reaction menstruum.

Reactive distillation has long been proposed for esterification reactions as water is continuously removed from the reaction menstruum. Although reactive distillation holds promise for the conversion of monoethylene glycol to ethylene glycol diacetate, the size and energy required for a traditional reactive distillation unit to achieve an ethylene glycol diacetate product with an acceptably low concentration of 2-hydroxyethyl acetate and water detracts from its economic viability.

Suman, et al., in Entrainer Based Reactive Distillation for Esterification of Ethylene Glycol with Acetic Acid, Ind. Eng. Chem. Res, 2009, 48, 9461-9470, discuss the use of an entrainer, ethylene dichloride, in a continuous reactive distillation column. In addition to facilitating the removal of water from the reactive distillation column, the ethylene dichloride entrainer is said to enable achieving complete separation of acetic acid from water. The mole fraction of ethylene dichloride in the reactive distillation column was about 0.5. The sizing of a reactive distillation column and heat duty for a commercial scale unit would have to take into account the large volume of the entrainer. Additionally, the entrainer serves to dilute the reactants and thus adversely affects the esterification reaction rates.

In 2016, Huang, et al., in Innovative Ethylene Glycol Diacetate Synthesis Process in a Single Reactive Distillation Column, Chemical Engineering and Processing, 109, 80-89, confirmed that it is not easy to obtain adequate purity of ethylene glycol diacetate by the esterification of monoethylene glycol with acetic acid. Huang, et al., disclose a proposed reactive distillation operation for making ethylene glycol diacetate from ethylene oxide and monoethylene glycol. Ethylene oxide is reported to react with water to make ethylene glycol, and the authors state in section 5.1 that the esterification of ethylene glycol and the hydration of ethylene oxide “might promote each other”. Huang, et al., conclude, based upon computer simulations, that with ethylene oxide as the feed, high purity and yield of ethylene glycol diacetate can be reached with lower equipment costs and energy requirements. Ethylene oxide, however, is not a favored reactant for commercial operations unless proximate to a plant making ethylene oxide, and such plants typically use ethylene from fossil sources, not sustainable resources.

Accordingly, an interest remains to make ethylene glycol diacetate from monoethylene glycol, preferably derived from sustainable resources, on an economically feasible basis, where the ethylene glycol diacetate is of sufficient purity for use as a product or intermediate, especially as an intermediate for making vinyl acetate monomer.

### BRIEF SUMMARY

Disclosed here are processes for making ethylene glycol diacetate from monoethylene glycol and acetic acid by reactive distillation are provided that can provide enhanced economics and advantageous ethylene glycol diacetate product quality, especially as an intermediate for the production of vinyl acetate monomer. In some embodiments, the processes for making ethylene glycol diacetate are integrated with processes for cracking of ethylene glycol diacetate to make vinyl acetate monomer.

In various embodiments, the reactive distillation is operated to provide a crude product containing ethylene glycol diacetate and 2-hydroxyethyl acetate, in some embodiments at least about 0.1 mass parts of 2-hydroxyethyl acetate per 100 mass parts of ethylene glycol diacetate, optionally at least about 0.2 mass parts of 2-hydroxyethyl acetate per 100 mass parts of ethylene glycol diacetate. In certain embodiments, the mass parts of 2-hydroxyethyl acetate per 100 mass parts of ethylene glycol diacetate can be between about 0.35 and about 5. In some embodiments, the mass parts of 2-hydroxyethyl acetate per 100 mass parts of ethylene glycol diacetate can be between about 0.5 and about 5. The crude product optionally contains water.

The crude product is contacted with acetic anhydride under conditions to provide a treated product having a reduced mass ratio of 2-hydroxyethyl acetate to ethylene glycol diacetate of less than about 0.1, according to some embodiments. In various embodiments, the reduced mass ratio of 2-hydroxyethyl acetate to ethylene glycol diacetate can be less than about 0.05. In some embodiments, the water in the treated product is less than about 0.05 mass parts per 100 mass parts of ethylene glycol diacetate. In further embodiments, the water in the treated product is less than about 0.001 mass parts per 100 mass parts of ethylene glycol diacetate.

The ability to operate the reactive distillation to provide a crude ethylene glycol diacetate product having a significant content of 2-hydroxyethyl acetate provides the disclosed processes capital and energy savings potential as compared to operating the reactive distillation to achieve the same ethylene glycol diacetate product composition through reactive distillation. Often 2 or more distillation stages can be eliminated and/or reboiler heat duty reduced without adversely affecting ethylene glycol diacetate product quality. Additional benefits that can be realized from the disclosed processes include (i) a reduction in the generation of heavies, especially where a homogeneous acid catalyst is used for the esterification and (ii) the ability to use strongly acidic, homogeneous acid catalysts for the esterification, and convert the catalysts by contact with acetic anhydride, to less acidic species thus reducing the corrosivity of the ethylene glycol diacetate product. Without wishing to be bound by theory, it is believed that heavies are formed under the conditions in the bottom section of the distillation column through reaction involving 2-hydroxyethyl acetate. The reduction in the content of 2-hydroxyethyl acetate by the treatment of the crude ethylene glycol diacetate product with acetic anhydride, provides a bottoms fraction having enhanced stability from the production of heavies. Additionally, the reduction in the generation of heavies reduces the volume of the purge that needs to occur, and facilitates the recovery of ethylene glycol diacetate from the purge.

Where the production of ethylene glycol diacetate is integrated with a cracking unit operation to make vinyl acetate monomer, the ethylene glycol diacetate passed to the cracking unit operation may contain at least about 0.5, and optionally between about 1 and about 10, such as, between about 2 and about 5, mass parts of acetic acid per 100 mass parts of ethylene glycol diacetate. The acetic acid can be added to the ethylene glycol diacetate being passed to the cracking unit operation and/or can be acetic acid which is in excess of that reacted in the reactive distillation. The use of stoichiometric excess of acetic acid in the reactive distillation may assist in driving the equilibrium reactions to ethylene glycol diacetate. Under cracking conditions, it has been found that a substantial portion of this co-fed, acetic acid is converted to acetic anhydride, which can be recovered for use in treating the crude ethylene glycol diacetate product. Hence, the acetic acid conversion efficiency to ethylene glycol diacetate can approach nearly 100 percent, and the requirements for acetic anhydride for treating the crude ethylene glycol diacetate product can be satisfied by an internal supply.

Various embodiments pertain to continuous processes for making ethylene glycol diacetate from monoethylene glycol and acetic acid by reactive distillation comprising:
a. continuously supplying to a reactive distillation zone, acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having a reduced mass ratio of 2-hydroxyethyl acetate to ethylene glycol diacetate; and
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone.

The distillation apparatus for conducting the disclosed processes typically comprises an upper, substantially non-reactive rectifying section to remove water from acetic acid which section is substantially devoid of esterification catalyst, a reactive distillation zone section (reactive distillation zone) containing esterification catalyst and a lower bottoms section, which may or may not be within the reactive distillation zone. The bottoms section is considered within the reactive distillation zone if it contains catalyst for esterification. In one option, the crude ethylene glycol diacetate product is contacted with acetic anhydride in a lower portion of the reactive distillation zone to provide the treated product. In another option, the crude ethylene glycol diacetate product is contacted with acetic anhydride in the bottoms section which is not within the reactive distillation zone to provide the treated product. In yet another option, the crude ethylene glycol diacetate is withdrawn from the bottoms section and contacted with acetic anhydride to provide the treated product. In some embodiments, the contacting of the acetic anhydride with the crude ethylene glycol diacetate product is in the presence of esterification catalyst. It may be beneficial to contact the crude ethylene glycol diacetate product with acetic anhydride where the acetic acid generated is capable of being readily passed to the reactive distillation zone.

The esterification catalyst can be any suitable esterification catalyst and can be heterogeneous or homogeneous. In some embodiments, the esterification catalyst is acidic and has a pKa of less than about 3, and in certain embodiments less than −1. In certain embodiments, the esterification catalyst comprises at least one of sulfuric acid and sulfoacetic acid.

In another embodiment, continuous processes for making vinyl acetate monomer from monoethylene glycol and acetic acid comprise:
a. continuously supplying to a reactive distillation zone, acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having reduced mass ratio of 2-hydroxyethyl acetate to ethylene glycol diacetate;
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone;
e. continuously passing at least a portion of the treated product to a cracking zone maintained under conditions sufficient to convert at least a portion of the ethylene glycol diacetate to vinyl acetate monomer, wherein acetic acid in an amount of at least 0.5 mass parts per 100 mass parts of ethylene glycol diacetate is also passed to the cracking zone, to provide a cracker effluent comprising vinyl acetate monomer and acetic anhydride;
f. continuously withdrawing cracker effluent from the cracking zone and selectively separating at least a portion of the acetic anhydride from the cracker effluent; and
g. passing at least a portion of the separated acetic anhydride to step (c).

The portion of the treated product being passed to the cracking zone in step (e) can be an aliquot portion, but typically is an aliquant portion from which at least a portion of one or more components in the treated product have been removed, e.g., heavies, acetic acid, and homogeneous catalyst, if used.

The cracker effluent also contains acetaldehyde acetaldehyde may be selectively recovered from the cracker effluent; at least a portion of the acetaldehyde is contacted with acetic anhydride under reaction conditions to provide 1,1-diacetoxyethane-containing product. If desired, at least a portion of the 1,1-diacetoxyethane is subjected to conditions sufficient to convert it to vinyl acetate.

This aspect may include, but is not limited to, continuous processes for making vinyl acetate monomer from monoethylene glycol and acetic acid comprising:
a. continuously supplying to a reactive distillation zone, acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having a reduced concentration of 2-hydroxyethyl acetate;
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone;
e. continuously passing at least a portion of the treated product to a cracking zone maintained under conditions sufficient to convert at least a portion of the ethylene glycol diacetate to vinyl acetate monomer, wherein acetic acid in an amount of at least 0.5 mass parts per 100 mass parts of ethylene glycol acetate is also passed to the cracking zone, to provide a cracker effluent comprising vinyl acetate monomer, acetaldehyde and acetic anhydride;
f. continuously withdrawing cracker effluent from the cracking zone and selectively separating at least a portion of the acetic anhydride from the cracker effluent and at least a portion of the acetaldehyde from the cracker effluent; and
g. contacting at least a portion of the separated acetic anhydride with at least a portion of the separated acetaldehyde under reaction conditions to produce a reaction product comprising 1,1-diacetoxyethane.

The reaction product of step (e) may be passed to step (c) and at least a portion of the 1.1-diacetoxyethane is converted to vinyl acetate.

While multiple embodiments are disclosed, still other embodiments of the disclosure will become apparent to those skilled in the art from the following detailed description, which shows and describes illustrative embodiments. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1 is a schematic depiction of an apparatus that can be used in conducting various disclosed processes, according to certain embodiments.

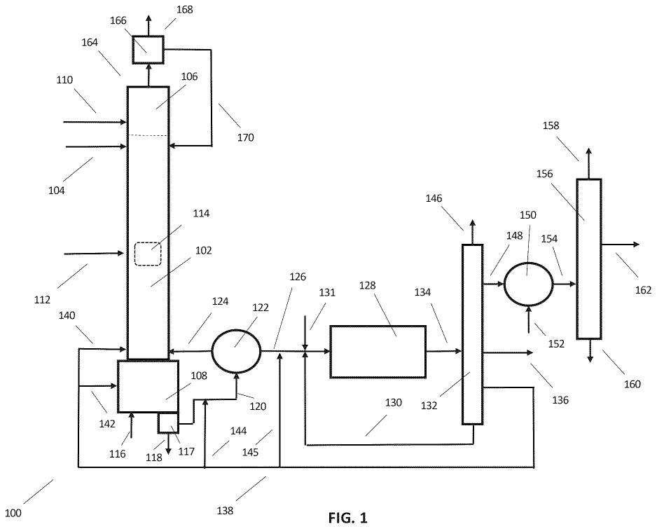

### DETAILED DESCRIPTION

All patents, published patent applications, and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3, 0.63, 0.815, and so forth) are included as are all intermediate ranges (e.g., 0.2-0.5, 0.54-0.913, and so forth).

The use of the terms “a” and “an” is intended to include one or more of the elements described.

Admixing or admixed means the formation of a physical combination of two or more elements which may have a uniform or non-uniform composition throughout and includes, but is not limited to, solid mixtures, solutions and suspensions.

Acetic acid providing feed is a feed containing acetic acid and/or precursors to acetic acid that will form acetic acid under esterification conditions such as acetic anhydride.

The term “Available Hydroxyls” means the hydroxyls on C2 to C4 hydrocarbons such as monoethylene glycol, 2-hydroxyethyl acetate, 1,2-propanediol, 1,2-butanediol, and the like.

A crude ethylene glycol diacetate product is an ethylene glycol diacetate-containing mixture at a point or region in the process and may or may not be recovered or isolated as a discrete product. Thus, it can be a point or region in a flowing stream such as the liquid phase in a distillation column, in the bottoms section of a distillation column or a flowing liquid phase in a reboiler.

A distillation plate or stage is a theoretical distillation plate ascertained by calculation. Where no reaction is occurring, the theoretical plate is where physical thermodynamic equilibrium is achieved between the vapor and liquid phases. Where a reaction is occurring, as in the reactive distillation zone, physical equilibrium is achieved but not chemical equilibrium as the rate of esterification slows approaching equilibrium. Often, at steady-state the composition of the liquid phase is at about 40 to 90 percent toward the equilibrium composition. For purposes of the reactive distillation zone, the physical thermodynamic equilibrium is for water and acetic acid in the vapor phase and in the liquid phase, and the chemical equilibria are for acetic acid, monoethylene glycol, 2-hydroxyethyl acetate and ethylene glycol diacetate in the liquid phase.

Ethylene glycol diacetate is also known as 1,2-diacetoxyethane.

The half acetate ester of ethylene glycol is also known as 2-hydroxyethyl acetate.

Hold-up is the quantity, or volume, of liquid phase that can be introduced into the reactive distillation zone without leaving the reactive distillation zone. The hold-up is influenced by the configuration of the internal structure of the zone. For instance, bubble trays will have a greater hold-up quantity than structured packing.

Monoethylene glycol is sometimes referred to herein as ethylene glycol.

A monoethylene glycol feedstock means a feedstock containing or derived from monoethylene glycol. Thus, the feedstock can contain monoethylene glycol with no 2-hydroxyethyl acetate or 2-hydroxyethyl acetate with no monoethylene glycol or mixtures of monoethylene glycol and 2-hydroxyethyl acetate.

Reactive distillation is a process in which reaction and separation occur simultaneously on some or all of the stages of a distillation column, and reaction products (e.g., ester and water) are separated and removed. The continuous removal of the reaction products via the distillation can increase conversion beyond that of the equilibrium composition in a batch reactor. Thus, as feedstock and esterification catalyst, which are introduced on an upper distillation stage of the reactive distillation zone, contact acetic acid rising in the vapor phase from a lower distillation stage where it has been introduced, the esterification reaction occurs, producing one or more acetate esters and water. The acetate esters, having a relatively high boiling point, remain mostly in the downward flowing liquid phase, whereas water, having a relatively low boiling point, mostly passes into the upward flowing vapor phase. As the liquid progresses downward to lower distillation stages of the reaction zone, 2-hydroxyethyl acetate and unreacted monoethylene glycol continue to react with the acetic acid rising from lower distillation stages, forming additional esters and water. The water, rising in the vapor from lower distillate stages, combines with water generated in the upper distillation stages. There being a vapor-liquid equilibrium relationship for all of the components, the water is present in the liquid phase in varying concentrations but tends to be present in lower concentrations in the liquid for lower distillation stages than it is in the upper distillation stages. Conversely, the concentration of acetic acid in both phases gradually decreases as it progresses upward in the column and is reacted away by the esterification reaction. The bottom (last reaction zone) of the distillation column can, therefore, have a liquid phase rich in ethylene glycol diacetate and very lean in water and 2-hydroxyethyl acetate, whereas the water leaving the top of the column (non-reactive rectification section) as distillate tends to have a low concentration of acetic acid.

As stated above, the distillation apparatus for conducting the disclosed processes may comprise an upper, substantially non-reactive rectifying section to remove water from acetic acid which section is substantially devoid of esterification catalyst, a middle reactive distillation zone section (reactive distillation zone) containing esterification catalyst and a lower bottoms section that receives the liquid phase from the fractionation section, which may or may not be a reactive zone depending upon the presence of esterification catalyst. The bottoms section, which also acts as a distillation stage with or without reactions occurring, can be heated to generate the vapor which passes to the bottom distillation stage of the fractionation section, or liquid can be withdrawn from the bottoms section and passed to a reboiler to heat the liquid and provide a vapor phase to be passed to the bottom distillation stage of the fractionation section.

Substantially non-reactive means that under the reaction conditions including time, temperature and presence of catalyst and other adjuvants, less than 1 percent of the moiety would be reacted.

Sustainable resources are plants and animals, including, but not limited to, waste products from plants and animals, and sustainable feedstocks are derived from sustainable resources. Sustainable resources also include carbon dioxide used as a feedstock in processes to make monoethylene glycol, whether that carbon dioxide is captured from direct air capture or from emissions from facilities that emit carbon dioxide, including, but not limited to, incineration, power generation, fermentations, and chemical and other industrial processes.

Vicinal glycols are 1,2-dihydroxyalkanes and lower vicinal glycols are monoethylene glycol, propylene glycol and 1,2-butanediol.

### Feedstocks

With respect to the esterification to produce ethylene glycol diacetate, in its broad aspects, the disclosed process contemplates the monoethylene glycol feedstock being generated from any process using any suitable raw material. Advantageously at least a portion of one or both of monoethylene glycol and acetic acid used to make ethylene glycol diacetate are derived from sustainable resources, and it may be that at least about 75 percent is derived from sustainable resources. The monoethylene glycol-containing feedstock can be derived from any suitable source. For instance, ethylene can be obtained from sustainable feedstocks and then converted to monoethylene glycol according to the conventional process of partial oxidation to ethylene oxide and hydrolysis to monoethylene glycol. The conventional process provides monoethylene glycol substantially free of propylene glycol and 1,2-butanediol. Alternative processes for making monoethylene glycol from sustainable feedstocks can have greater efficiencies of carbon in the biomass to monoethylene glycol but have challenges in securing high purities. For instance, a sustainable feedstock can be converted to syngas and monoethylene glycol can be made by the glycol oxalate process such as disclosed in U.S. Pat. No. 4,453,026. The glycol oxalate process also generates impurities including 1,2-butanediol. Carbohydrates can be converted to monoethylene glycol by retro aldol conversion of the carbohydrate to, among others, glycol aldehyde, and hydrogenation to monoethylene glycol (Retro Aldol Process). See, for instance, U.S. Pat. No. 9,783,472. This process can have a high selectivity to monoethylene glycol, but produces, among others, vicinal glycols including, but not limited to, propylene glycol and 1,2-butanediol. The separation of monoethylene glycol from other vicinal glycols can be energy intensive, especially 1,2-butanediol which has substantially the same normal boiling point as monoethylene glycol. Certain processes herein integrate with the production of monoethylene glycol from certain processes, such as the Retro Aldol Process and the glycol oxalate process, for purposes of removing propylene glycol and 1,2-butanediol by removing their reaction products.

The acetic acid, which may be derived from sustainable resources, such as produced by the fermentation of sugars, or fossil feedstocks, can be supplied to process in any suitable form including glacial acetic acid or aqueous solutions of acetic acid. The acetic acid feed may also contain acetic anhydride, but only in a minor amount, such as, up to 10 mass percent of the acetic acid feed to the reactive distillation is acetic anhydride. For purposes herein, the amount of acetic acid is based upon the theoretical moles of acetic acid that can be derived from the form of acetic acid supplied.

Although most conveniently, monoethylene glycol is fed to the reactive distillation zone, it is understood that a pre-reaction of monoethylene glycol with acetic acid can occur to convert at least some of the monoethylene glycol to 2-hydroxyethyl acetate prior to introduction into the reactive distillation zone. Water generated by the pre-reaction can remain in the pre-reaction product or removed at least in part. Thus, the feed to the reactive distillation can be monoethylene glycol, 2-hydroxyethyl acetate or a mixture thereof. The pre-reaction can be conducted in any suitable manner. Where a pre-reaction is conducted, the feed frequently contains between about 10 and 90, such as, 20 to 50, mole percent 2-hydroxyethyl acetate based upon total monoethylene glycol and 2-hydroxyethyl acetate. The pre-reaction product may optionally contain esterification catalyst and unreacted acetic acid, and these moieties are to be included in determining the feed amounts to the reactive distillation zone. Where a pre-reacted feed is used, the calculation of the amount of acetic acid to be provided to the reactive distillation zone is based on Available Hydroxyls.

### Reactive Distillation Process Conditions

The acetic acid is provided in at least a stoichiometric amount required to form the diacetate of the monoethylene glycol and diacetates of any other lower vicinal glycols in the feedstock. For purposes herein, where the esterification reaction system provides a feed to a cracking unit operation to make vinyl acetate, acetic acid, acetaldehyde and acetic anhydride that are generated by the cracking and recycled to the reaction system, are considered to be part of the acetic acid feed provided to the reaction system.

Usually, the total amount of acetic acid including acetic anhydride provided per unit time to the reaction system (excluding any acetic acid steady state inventory, that is, acetic acid which is retained in the reactive distillation zone) is less than about 1.5, such as, about 1 to 1.15, times that required on a stoichiometric basis to convert the Available Hydroxyls in the monoethylene glycol containing feedstock provided per that unit time to the corresponding acetate esters. The excess acetic acid will pass to the bottom of the reactive distillation zone and can, if desired, be recovered from the bottoms of the reactive distillation zone and recycled. Some, usually a very minor amount, of acetic acid can be contained in the water fraction from the overhead, and some can be contained in purge streams. As discussed herein, at least a portion of the unreacted acetic acid can be passed to a cracking unit operation and converted, in part, to acetic anhydride.

In an exemplary implementation, the reactive distillation zone contains at steady state operation, an inventory of acetic acid (“steady state inventory acetic acid”) which is in addition to the amount of acetic acid fed. The steady state inventory is primarily reflective of the liquid phase hold-up in the reactive distillation zone with an increase in hold-up resulting in an increase in the steady state inventory of acetic acid. Where the steady state inventory acetic acid results in a higher mole ratio of acetic acid to Available Hydroxyls in the liquid phase in a given distillation stage, the driving force for the esterifications is increased. Regardless, an increase in the liquid hold-up due to an increase in the amount of the steady state inventory acetic acid at any given distillation stage serves to increase the liquid residence time in that stage giving more time for the esterifications to proceed toward chemical equilibria. The amount of the steady state inventory acetic acid depends upon the configuration and operation of the reactive distillation as does the distribution of the steady state inventory acetic acid. Often, the steady state inventory acetic acid in the vapor phase and in the liquid phase in the reactive distillation zone as calculated as moles of acetic acid to moles of Available Hydroxyls in the feedstock is greater than 0.1:1 or 0.2:1, such as, about 0.25:1 to 2:1. It is to be understood that the concentration of acetic acid in any given distillation stage will be determined by the distillation operating parameters and can vary widely over the height of the reactive distillation zone. For example, this ratio may be as low as 0.0001:1 in the liquid phase at the top most distillation stage and as high as 100:1 in the liquid phase at the bottom most stage of the reactive distillation zone.

This steady state inventory acetic acid can be maintained in the reaction system by any suitable means, e.g., by using one or more of the following: optimizing the acetic acid feed location, increasing the boil-up rate in the column, retaining acetic acid in the reaction system through packing or tray design and recycling excess acetic acid recovered from the reaction system. For example, the use of, for example, bubble trays and valve trays, which can have large liquid hold-ups, in the reactive distillation zone can assist in maintaining the steady state inventory acetic acid. In general, the greater the hold-up in a distillation stage, the closer the liquid phase comes to equilibrium for the liquid composition at that stage. However, increased liquid hold-up usually comes with a penalty of increased pressure drop. Increasing catalytic activity also results in pushing the esterification reactions closer to equilibrium for the liquid composition at that stage, but costs associated with the use of more catalyst have to be taken into account. Hence, with higher catalytic activity, the hold-up can be reduced while obtaining substantially the same performance as with a higher hold-up. It is also to be understood that the design of the reactive distillation zone can have different sections with different hold-ups per distillation stage. For instance, distillation stages in the upper and the lower sections of the reactive distillation column generally have lower rates toward equilibrium than those in the intermediate section. Accordingly, the distillation stages in at least one of the upper section and the lower section of the reactive distillation zone could be designed to have a higher hold-up with the intermediate section having a lower hold-up, and thereby attenuate pressure drop but yet still obtain the same conversion to ethylene glycol diacetate. Frequently, the hold-up averages up to about 0.5, such as, about 0.01 to about 4, and more often about 0.5 to about 3, liters of liquid per kilogram per hour of monoethylene glycol in the feed per distillation stage.

The monoethylene glycol feedstock is subjected to acidic esterification conditions in the presence of acetic acid to provide ethylene glycol diacetate. Acidic esterification conditions include the presence of an acidic catalyst and elevated temperatures. Suitable esterification catalysts have a pKa of less than about 3, often less than about −1, and are not unduly reactive with monoethylene glycol. The esterification catalysts may be heterogeneous or homogeneous, and examples of catalysts include sulfonic membranes such as Nafion™ sulfonated tetrafluoroethylene-based fluoropolymer, solid Brønsted acids such as graphene supports having functional groups (one or more of SO3H−, COOH−, and phenolic OH−) thereon, methane sulfonic acid, p-toluene sulfonic acid, and trifluoromethanesulfonic acid. Esterification catalysts also include inorganic acids such as phosphoric acid, phosphonic acid, sulfuric acid, sulfonic acid, sulfoacetic acid and mixtures thereof. The catalyst is employed in a catalytically effective amount, generally for homogeneous catalysts, in the range of about 0.01 to 10 grams of catalyst per 100 grams of liquid phase in the reactive distillation zone. Heterogeneous catalysts are often provided such that the mass hourly space velocity of monoethylene glycol feed to mass of heterogeneous catalyst is in the range of about 0.01 to about 50, such as about 0.05 to about 20, hour-1. The heterogeneous catalyst can be distributed uniformly per each distillation stage in the reactive distillation zone, or non-uniformly such that at least one distillation stage contains a greater amount of heterogeneous catalyst than does at least one other distillation stage.

The esterification is conducted in the liquid phase within the reactive distillation zone. Vapor and liquid phases must be present on all distillation stages of the reactive distillation zone for the separations to occur, so, for a given column pressure, the temperature on each distillation stage will be the boiling point of the liquid mixture on that stage and is dependent upon the composition of that mixture. If an azeotroping agent (entrainer) is used, then the boiling points of the distillation stages where the azeotroping agent is present will be affected by the presence of the azeotroping agent. Thus, water, which is a coproduct of the esterification is removed as a vapor in the reactive distillation zone thereby driving the esterification reactions towards completion.

In some instances, a component that azeotropes with water is present and serves to reduce the temperature required to remove water. Examples of azeotroping agents for the dehydration of acetic acid are known, see, for instance, U.S. Pat. No. 5,160,412, and for the reactive distillation of ethylene glycol with acetic acid, are known, see, for instance Suman, et al., Entrainer Based Reactive Distillation for Esterification of Ethylene Glycol with Acetic Acid, Ind. Eng. Chem. Res., 2009, 48, 9461-9470. As will be discussed later, the use of azeotroping agents in the reactive distillation dilute the reactants and can increase capital and heat duty. In one aspect, an azeotroping agent, when used, is one that has a normal boiling point at least about 25° C. below the normal boiling point of acetic acid and thus the azeotroping agent is substantially not present in the reactive distillation column below the point of introduction of the azeotroping agent.

The esterification conditions typically include a pressure in the range of about 10 to 500, often 50 to 250, kilopascals absolute, and maximum temperatures in the reactive distillation (excluding the still bottoms and any reboiler that provide heat to the distillation column) are in the range of about 80° C. to about 250° C., such as, about 90° C. to about 200° C.

### Reactive Distillation Operation

It is to be understood that the reactive distillation zone may reside in a single column, which may be beneficial in some aspects, or two or more distillation columns, when project constraints require it. This disclosure, by providing a desirable ethylene glycol diacetate product having very low water and 2-hydroxyethyl acetate content through reaction with acetic anhydride, can result in the need for one or both of fewer distillation stages for the same ethylene glycol diacetate production rate and a lower heat duty since the reflux can be reduced, all else essentially the same. Thus, the ability to benefit from the use of a single reactive distillation column is facilitated by the presently disclosed processes.

The monoethylene glycol feedstock is typically provided to the highest distillation stage or stages containing catalyst. The portion of the distillation column above the point of introduction of the monoethylene glycol feedstock (non-reactive rectification section) is for primarily for the removal of acetic acid from water but also serves to remove monoethylene glycol and esters that pass to the non-reactive rectification section. If desired, a portion of monoethylene glycol of the feedstock, e.g., about 10 to 50 mass percent of the total monoethylene glycol feedstock, can be fed to the non-reactive rectification section below its top distillation stage. As the normal boiling point of monoethylene glycol is about 80° C. higher than that of acetic acid, it passes downwardly in the non-reactive rectification section as a liquid and back to the reactive distillation zone. This monoethylene glycol is believed to assist in pushing the acetic acid downwardly, and thus serves to reduce capital and heat duty for this portion of the distillation column.

The acetic acid feedstock can be introduced at any location or at two or more locations in the reactive distillation zone. In some embodiments, it may be beneficial that the location(s) of acetic acid introduction be sufficiently high in the reactive distillation zone to enhance the rate of conversion to the esters, but not so high as to result in the loss of undue amounts of acetic acid in the overhead from the nonreactive rectification section. Where a single feed point is used, the location of the acetic acid introduction is at a distillation stage having a mass ratio of ethylene glycol to 2-hydroxyethyl acetate in the liquid phase in the range of about 0.5:1 to about 10:1, and especially where the feedstock is monoethylene glycol, in the range of about 1:1 to about 6:1, such as, 1.5:1 to about 5:1. The use of two or more acetic acid feed locations is disclosed in copending United States provisional patent application 9022034-194048, filed on even date herewith. Where two or more feeds to the reactive zone are used, at least one of heat duty and capital costs for the reactive distillation are reduced for a given conversion to ethylene glycol diacetate: 2-hydroxyethyl acetate ratio as compared to all the acetic acid feed to a single distillation stage. Surprisingly, the use of two or more locations of acetic acid feed can provide a lower heat duty than if the feed were only provided to a single, intervening distillation stage. The use of two or more locations of acetic acid feed can be implemented to alter the acetic acid concentrations in the liquid phase as compared to a single introduction location, yet still achieve desirably low amounts of acetic acid passing into the overhead. In some embodiments, at least about 40 percent of the acetic acid is fed to the lower section of the reactive distillation zone, and various embodiments at least about 20 percent of the acetic acid is fed to the middle section of the reactive distillation zone. Often, one or more of the distillation stages in the middle section to which a portion of the acetic acid fed have a liquid phase in which the mole ratio of ethylene glycol diacetate to 2-hydroxyethyl acetate between about 0.01:1 and 20:1. Frequently, between about 40 and 80 percent of the acetic acid is fed to the lower section of the reactive distillation zone, and the feed location is at least one of the bottom three distillation stages.

The separation of water from acetic acid in the vapor phase passing from the reactive distillation zone is accomplished by fractional distillation in the non-reactive rectification section of the column. The number of distillation stages used will, in part, be determinative of the acceptable concentration of acetic acid in the water overhead. Since acetic acid is readily susceptible to degradation in wastewater treatment facilities, the primary consideration of the operator is the cost of the lost acetic acid and the energy and capital costs for the water and acetic acid separation. In general, the concentration of acetic acid in the water overhead may be less than about 2, and may be up to about 1 mass percent.

The use of azeotropic distillation has been proposed to achieve enhanced separations. See, for instance, Suman, et al., discussed above. In various aspects of the processes of this disclosure, certain azeotroping agents, or entrainers, are present in the non-reactive rectification section of the reactive distillation column. Various relevant entrainers are characterized as having a normal boiling point of less than about 115° C., a normal azeotropic minimum boiling point no greater than about 80° C., a mutual solubility of water in the entrainer (at 25° C.) of less than about 5 mole percent, a mutual solubility of the entrainer in water of less than about 5 mole percent, and a substantial lack of reactivity in the reaction menstruum in the reactive distillation column. Examples of entrainers include, but are not limited to, heptane, ethylene dichloride, cyclohexane, benzene, di-isopropyl ether, and hexane. In this aspect, the entrainer is introduced into the reactive distillation column at or above the distillation stage to which the monoethylene glycol feedstock is introduced where it rapidly becomes vaporized either as itself or as an azeotrope. Thus, the entrainer does not unduly dilute the reactants in the reactive distillation portion of the distillation column nor require undue increases in heat duty. The water and azeotrope in the overhead can be condensed, and via liquid phase separation, an aqueous phase can be directed to wastewater treatment and the organic liquid phase can be recycled to the reactive distillation column.

The reactive distillation zone provides as a product, a liquid phase having acetic acid and ethylene glycol diacetate as the predominant components, frequently together they comprise at least about 90, such as, between about 95 to about 99.9, mass percent of the liquid phase. This liquid phase can also contain 2-hydroxyethyl acetate and water until acetic anhydride is added, and it can contain homogeneous esterification catalyst, if used, until contact with acetic anhydride.

The disclosed processes can be operated such that appreciable 2-hydroxyethyl acetate still remains in an intermediate esterification product stream (that is the liquid phase containing ethylene glycol diacetate immediately before it is contacted with acetic anhydride in accordance with this disclosure). This intermediate esterification product stream is also referred to herein as the crude diethylene glycol diacetate product. The operator has the flexibility to adjust the concentration of 2-hydroxyethyl acetate in the esterification product primarily by one or more of (i) setting the number of distillation stages in the reactive distillation zone, with fewer resulting in increased 2-hydroxyethyl acetate concentration, (ii) setting the reflux ratio, with lower reflux ratios resulting in increased 2-hydroxylethyl acetate concentration, (iii) setting the hold-up in the reactive distillation zone, with lower hold-up, i.e., reduced steady state inventory acetic acid, resulting in increased 2-hydroxyethyl acetate concentration. In general, the conditions providing a higher concentration of 2-hydroxyethyl acetate in the esterification product beneficially provide one or more of capital cost and heat duty cost savings. The concentration of 2-hydroxyethyl acetate in the crude esterification product before contact with acetic anhydride is often at least about 0.2, such as, between about 0.35 or about 0.5 and about 5, and frequently between about 1 and 4, mass parts per 100 mass parts of ethylene glycol diacetate. The concentration of water in the crude esterification product prior to contact with acetic anhydride may be less than about 0.5, and optionally less than about 0.1 or 0.2, mass parts per 100 mass parts of ethylene glycol diacetate.

The liquid phase from the fractional distillation section of the reactive distillation column (which may or may not have been contacted with acetic anhydride) is typically passed to a bottoms section or kettle. The bottoms section can be heated to provide a heated vapor stream to pass into the reactive distillation zone, provide the heat energy for the distillation, and provide a residual liquor containing ethylene glycol diacetate. Alternatively, the liquid phase can be passed to a reboiler to provide the heated vapor phase to be returned to the reactive distillation zone. In either event, a significant portion of the acetic acid may be returned to the reactive distillation zone.

### Acetic Anhydride Addition

In accordance with this disclosure, acetic anhydride is contacted with a crude ethylene glycol diacetate product to provide a treated product having a lower concentration of 2-hydroxyethyl acetate and a lower concentration of water. Generally, the crude ethylene glycol diacetate contains water, and the amount of water will depend upon the location in the process where the contact with the acetic anhydride occurs as will be discussed later.

Under the conditions of the contacting, acetic anhydride reacts with 2-hydroxyethyl acetate to produce ethylene glycol diacetate and acetic acid, and with water to generate acetic acid. If homogeneous acid esterification catalyst is present, acetic anhydride will also react with the catalyst, also generating acetic acid. The molar ratio of acetic anhydride to the total moles of 2-hydroxyethyl acetate and water, is sufficient to reduce the concentration of 2-hydroxyethyl acetate and water in the treated ethylene glycol diacetate product to desired levels, e.g., to less than about 0.1, such as, less than about 0.05 mass parts of 2-hydroxyethyl acetate per 100 mass parts of ethylene glycol diacetate, and less than about 0.05, such as less than about 0.01, mass parts of water per 100 mass parts of ethylene glycol diacetate. In any event, the amount of acetic anhydride provided may be sufficient to reduce the amount of 2-hydroxyethyl acetate by at least about 50, optionally at least about 75, percent. In general, the amount of acetic anhydride introduced is in a mole ratio to the 2-hydroxyethyl acetate in the crude ethylene glycol diacetate product, of at least about 0.4:1, such as at least about 0.5:1, and often between about 1:1 to 10:1. Where homogeneous acid esterification catalyst is used, the amounts of acetic anhydride used may need to be adjusted to reflect reaction with the catalyst.

The conditions of contacting are advantageously those existing in the region into which the acetic anhydride is introduced, which, as discussed below can be at several points in the process. The reaction of acetic anhydride with 2-hydroxyethyl acetate proceeds quickly, especially in the presence of acid catalyst. Hence, the existing conditions in the reactive distillation zone or bottoms section or a liquid transport line between the two, where the contact with the acetic anhydride occurs, are usually sufficient to quickly provide a reduced mass ratio of 2-hydroxyethyl acetate to ethylene glycol diacetate. Unreacted acetic anhydride, which often is that amount in excess of the stoichiometric amount to react with 2-hydroxyethyl acetate, water and acid catalyst, can be recycled for use in the process. Acetic anhydride has a normal boiling point of about 140° C., which is lower than that of ethylene glycol diacetate (about 186° C.), and thus, if desired, can be separated as a vapor and passed to the reactive distillation zone as a source of acetic acid, especially where adequate water is present at the point of introduction to react with the acetic anhydride, or as a portion of the acetic anhydride used to contact the crude ethylene glycol diacetate product.

The acetic anhydride can be from any suitable source. In the embodiments where the conversion of monoethylene glycol to ethylene glycol diacetate is integrated with a cracking operation to make vinyl acetate monomer from the ethylene glycol diacetate, acetic anhydride is made in the cracking operation.

The acetic anhydride can be introduced at one or more points in the process with the objective of providing an ethylene glycol diacetate product that has a very low concentration of 2-hydroxyethyl acetate and water. Four of the most typical locations of introduction of the acetic anhydride are discussed below. It may be beneficial that the acetic anhydride is quickly dispersed in the crude ethylene glycol diacetate stream. For example, but not in limitation, the acetic anhydride can be provided to a distillation stage within reactive distillation zone and/or the bottoms section of the column or reboiler and/or an in-line mixer for contacting crude ethylene glycol diacetate.

In one option of practicing the disclosed processes, at least a portion of the acetic anhydride is introduced into a lower portion of the reactive distillation zone and above the bottoms section. In various embodiments, this may occur at a distillation stage where, absent to introduction of the acetic anhydride, the liquid phase would have contained less than about 5, and sometimes between about 0.1 to 3 or 4, mass parts 2-hydroxyethyl acetate per 100 mass parts ethylene glycol diacetate. Since acetic anhydride also reacts with water, introduction of the acetic anhydride at a point where the water concentration is already low can be beneficial. Often the liquid phase at the distillation stage to which acetic anhydride is introduced would, absent the introduction of the acetic anhydride, contain less than about 0.5, and sometimes less than about 0.1, mass parts water per 100 mass parts ethylene glycol diacetate. Although the reaction of 2-hydroxyethyl acetate and acetic anhydride proceeds relatively quickly, especially in the presence of esterification catalyst, in some instances the introduction of the acetic anhydride is at a distillation stage at least one above the bottom distillation stage to increase time for admixing and reaction. It should be understood that not all the 2-hydroxyethyl acetate need be reacted before the liquid phase passes to the bottoms section of the distillation column as the reaction between the acetic anhydride and 2-hydroxyethyl acetate can continue in the bottoms section.

In another option, at least a portion of the acetic anhydride is introduced into the bottoms section of the distillation column. Absent the introduction of water into or its generation within the bottoms section, the bottoms section typically has a liquid phase having the lowest concentration of water in the distillation column. Hence introducing the acetic anhydride at this location is beneficial since less acetic anhydride will be lost to reaction with water. Moreover, if homogeneous esterification catalyst is used, contacting in in a downcomer between the bottom most tray of the reactive distillation zone and the bottoms section enhances the reaction in the bottom most tray to proceed toward chemical equilibrium as the catalyst remains active, yet the catalyst is rendered less acidic before entering the bottoms section, thereby reducing the corrosiveness of the treated ethylene glycol diacetate product.

Where a reboiler is used that is separate from the bottoms section of the distillation column, a third option is to introduce the acetic anhydride into the reboiler. Where a homogeneous esterification catalyst is used, it may be beneficial for it to be first removed or converted to a less acidic species such as disclosed later.

The mass ratio of acetic acid to ethylene glycol diacetate in the ethylene glycol diacetate product can be varied depending upon the use for the ethylene glycol diacetate. Distillation can be sufficient to reduce the mass ratio of acetic acid to ethylene glycol diacetate to less than 1, and sometimes less than 0.1, parts per 100 parts of ethylene glycol diacetate. Where the ethylene glycol diacetate is to be converted to vinyl acetate monomer, the presence of acetic acid may be desired to generate acetic anhydride for use in the process, for instance, in amounts up to about 10 or 15 parts by mass of acetic acid per 100 parts by mass, of ethylene glycol diacetate.

A fourth option is to remove from the distillation system a crude ethylene glycol diacetate product, which may be liquid or vaporous, and contact it with acetic anhydride. This contacting will generate acetic acid from 2-hydroxyethyl acetate. Where the treated ethylene glycol diacetate is subjected to cracking conditions, the presence of acetic acid is not unduly adverse to the cracking operation, and can provide a source of acetic anhydride as will be discussed later. One embodiment of this option contemplates conducting the contacting the crude ethylene glycol diacetate product with acetic anhydride in the vapor phase. Maintaining the crude ethylene glycol diacetate and the treated product in the vapor phase can be advantageous to avoid the costs to revaporize the treated product for the cracking operation. On the other hand, if the cracking is conducted at a higher pressure, this advantage can be offset by compression costs. If desired, the vapor phase contacting can be conducted in the presence of solid acidic catalyst, such as Nafion™ resin available from the Chemours Company, Wilmington, Delaware, which has a tolerance to temperatures of 190° C., which temperature is above the normal boiling point of ethylene glycol diacetate.

Acetic anhydride can react with many acid esterification catalysts to provide acetic acid and an acetate of the catalyst. Certain embodiments of the disclosed processes may use sulfuric, sulfoacetic or phosphoric acid as the acid catalyst. Sulfuric acid, sulfoacetic and phosphoric acid are soluble in the reaction menstruum and pass downwardly in the distillation column. The addition of acetic anhydride to the reactive distillation zone or in the bottoms section in excess of that which eliminates water (e.g., to a concentration below about 50, and sometimes below about 20, parts per million by mass based upon the mass of the ethylene glycol diacetate) results in the formation of an acylated sulfate or sulfoacetic acid or phosphate species, as the case may be. The chemical composition of the acylated sulfate or sulfoacetic acid or phosphate species is not known, and the reaction product may, or may not, contain an acyl moiety. Nevertheless, a reaction occurs and for sake of convenience, the reaction product is referred to herein as an acylated species. These acylated species remain in the liquid phase at temperatures suitable for removing ethylene glycol diacetate from the acylated catalysts. If desired, the acylated catalysts can be used for esterification. Advantageously, the acylated catalysts have less acidity than do the acids, and thus materials of construction need not have the same resistance to corrosion as is required for contact with the acids themselves. The acylation of acid catalysts is disclosed in more detail in copending United States provisional patent application 9022034-194047, filed on even date herewith.

### Integration With Cracking to Produce Vinyl Acetate Monomer

Due to the significant commercial demand for vinyl acetate monomer, the ability to integrate the reactive distillation to convert monoethylene glycol to ethylene glycol diacetate and a cracking operation may be commercially important. Accordingly, in addition to ethylene glycol diacetate, acetic acid may be fed to a cracking zone. The cracking zone may be a catalytic cracking zone but is conveniently a thermal cracking zone. Often thermal cracking zones may be operated at temperatures between about 475° C. and 600° C., and in some embodiments, between about 490° C. and 550° C. The thermal cracking has an inverse relationship between conversion and selectivity to vinyl acetate monomer. Hence, the temperature of the cracking zone and the residence time of the gases in the cracking zone can be adjusted to achieve a desired balance between conversion and selectivity. In various embodiments, the conversion of the ethylene glycol diacetate is between about 10 and 35, optionally between about 15 and 25, percent to provide a selectivity to vinyl acetate monomer over 80 percent. Typically, the gases in the cracking zone are at a high space velocity, e.g., at least 1000, and sometimes between about 1500 and 50000, kilograms per hour per square foot.

Where the ethylene glycol diacetate feed to the cracking zone contains acetic acid, under the conditions of the cracking, a material increase the amount of acetic anhydride in the cracking effluent occurs, and the acetic anhydride can be recovered and, at least in part, passed as the acetic anhydride reactant to the reactive distillation system. Optionally a portion of the acetic anhydride can be reacted with acetaldehyde from the cracker effluent. In one aspect of the disclosed processes, acetaldehyde is selectively recovered from the cracker effluent and is contacted under reaction conditions with acetic anhydride to make 1,1-diacetoxyethane. The 1,1-diacetoxyethane can be recycled to the cracking zone or subjected to another unit operation to make vinyl acetate. Any suitable process can be used to make the 1,1-diacetoxyethane including those disclosed in U.S. Pat. No. 2,021,698 which may also generate vinyl acetate. In general, to make 1,1-diacetoxyethane, the acetic anhydride and acetaldehyde are contacted at elevated temperature, e.g., in the range of about 70° to 200° C., with or without the presence of catalyst. Catalysts include metal halide catalysts, especially ferric chloride, and sulfuric or phosphoric acid catalysts. The acetic anhydride and acetaldehyde can be reacted in a dedicated reaction zone or alternatively admixed with the feed to the cracking zone and reacted in situ to make 1,1-diacetoxyethane. As can be readily appreciated, the selectivity of the raw materials to vinyl acetate monomer can be enhanced.

The acetaldehyde can also be oxidized to acetic acid or acetic anhydride. See, for instance, U.S. Pat. No. 4,094,901A and U.S. Pat. No. 4,252,983. As these processes involve the presence of oxygen, the removal of oxygen should be assured prior to recycling the acetic acid or acetic anhydride to the process.

### Drawing

Reference is made to the drawing which is provided to facilitate the understanding of the instantly disclosed process but is not intended to be in limitation of the disclosed process. The drawing is a schematic depiction of an apparatus generally designated as 100 suitable for practicing the described processes. The drawing omits ancillary unit operations and omits minor equipment such as pumps, heat exchangers, valves, instruments and other devices and unit operations the placement of which and operation thereof are well known to those practiced in chemical engineering.

Monoethylene glycol feedstock is supplied to reactive distillation column 102 via line 104. The monoethylene glycol feedstock may be pre-reacted in whole or part to 2-hydroxyethyl acetate. For purposes of discussion, no pre-reaction is effected. Column 102 contains esterification catalyst which can be homogeneous or heterogeneous. For purposes of discussion, a homogeneous catalyst, sulfuric acid, is used and is introduced admixed with the monoethylene glycol feedstock. The combination of the temperature and of the residence time of the mixture is maintained below that at which undue amounts of 1,4-dioxane is formed. In some embodiments, the catalyst is mixed with the monoethylene glycol proximate in time to the introduction of the monoethylene glycol into reactive distillation column 102. Typically, the temperature of the monoethylene glycol and catalyst feed is about the temperature of the boiling point of acetic acid under the conditions at the distillation stage of introduction.

Reactive distillation column 102 has a reactive distillation zone 114 with a non-reactive rectification zone 106 there above and a bottoms section 108 there below. Note that if catalyst is active in the bottoms section, the bottoms section would be included as a distillations stage in the reactive distillation zone. As depicted, line 110 directs a portion of the monoethylene glycol without catalyst into the non-reactive rectification zone to assist in pushing acetic acid down the column. Acetic acid feedstock is passed to column 102 via line 112 into a middle section of reactive distillation zone 114. The conversion to ethylene glycol diacetate increases toward the bottom of the column 102, and a crude ethylene glycol diacetate product is provided. The generation of a treated ethylene glycol diacetate product will be discussed later. For purposes of discussion at this point of the description, the liquid phase passing to the bottoms section 108 shall be referenced as the bottoms liquid.

In the absence of supplying acetic anhydride to the reactive distillation or the bottoms section, the bottoms liquid will contain homogeneous acid catalyst. In one option, a basic neutralizing agent is supplied to bottoms section 108 via line 116 to neutralize the catalyst and provide a solid precipitate which is filtered in filtration unit operation 117, and the filter cake is withdrawn via line 118. Advantageously the residence time of the bottoms liquid before neutralization of the acid catalyst is minimized to attenuate the occurrence of undesired reactions of the ethylene glycol diacetate.

Alternatively, where acetic anhydride is added to the reactive distillation zone or bottoms section, the acetic anhydride can convert the catalyst to a less acidic, acylated sulfate species, and the use of a basic neutralizing agent and equipment as described in the preceding paragraph is usually not necessary. The acylated sulfate species can be withdrawn as a liquid phase from still bottom section 108 via line 118. Line 118 can also serve as a liquid purge. The liquid purge can be subjected to vacuum evaporation, e.g., in a wiped film evaporator, to recover ethylene glycol diacetate as a vapor which can be returned to the process and provide a concentrated heavies phase. The acylated sulfate species remains as a liquid during the evaporation even though the liquid purge is concentrated to 5 mass percent or less of the original purge stream. If desired, the concentrated heavies can be subjected to extraction with water to provide an aqueous phase rich in the acylated sulfate species or a hydrolysis product thereof and an organic phase having reduced acylated sulfate species content. Ethylene glycol diacetate will be in the purge and can be readily recovered by contacting with water to provide an azeotrope of water and ethylene glycol diacetate that can be removed by vaporization from the purge stream.

The bottoms liquid having little or no catalyst due to neutralization or acetic anhydride treatment is passed as a liquid via line 120 to reboiler 122. It should be understood that reboiler 120 can be a pump-around reboiler with liquid being returned from the reboiler to the bottoms section where it is flashed. The vapor phase can, if desired, be passed via line 124 to a lower portion of column 102 to provide heat for the distillation, and the liquid phase is passed via line 126 to cracking reactor 128. Often the liquid phase contains up to about 99 percent of the ethylene glycol diacetate fed to reboiler 122. In some embodiments, the liquid phase contains between about 50 and about 98 percent of the ethylene glycol diacetate fed to reboiler 122 Also, in some instances, sufficient acetic anhydride is fed to reboiler 122 via line 117 to maintain any 2-hydroxyethyl acetate that forms by degradation of ethylene glycol diacetate, at a desirably low concentration. If desired, the aqueous phase containing the acylated sulfate species or hydrolysis product thereof, can be recycled as catalyst to the reactive distillation zone.

In the event that the liquid in line 126 does not contain sufficient acetic acid to generate the desired amount of acetic anhydride during the cracking, acetic acid can be provided via line 131. As the cracking in cracking reactor 128 typically only converts a portion of the ethylene glycol diacetate to vinyl acetate monomer, unreacted ethylene glycol diacetate is recovered from the cracking reactor effluent and recycled to the cracking reactor. As shown, recycled ethylene glycol diacetate is introduced into line 126 by line 130. Alternatively, the recycled ethylene glycol diacetate can be directly introduced into cracking reactor 128, and if desired, acetic acid can be supplied to the recycling ethylene glycol diacetate.

Cracking reactor 128 is, for purposes of this discussion, a tube in shell, vapor-phase, thermal cracking reactor operating at about 525° C. with a residence time sufficient to provide an effluent where about 18 percent of the ethylene glycol diacetate has been converted with a selectivity to vinyl acetate monomer on a mass basis of about 87 percent. The effluent, in addition to vinyl acetate monomer and ethylene glycol diacetate, usually contains acetic acid, acetic anhydride, 2-hydroxyethyl acetate, acetaldehyde, propionaldehyde, butyraldehyde, acetone, and butyrolactone. The effluent passes to distillation column 132 via line 134. Distillation column 132 can be one or more columns. A heavy boiling ethylene glycol diacetate fraction is withdrawn from column 132 via line 130 for recycle. Not shown is a heavies purge from this line. If desired, the heavies purge can be subjected to fractional distillation to recover ethylene glycol diacetate from the heavies. As the cracking of ethylene glycol diacetate results in the generation of acetic acid, acetic acid is withdrawn from column 132 via line 136 and can be recycled to distillation column 102 via line 112 and, if needed, to line 131. Also withdrawn from column 132 is an acetic anhydride fraction via line 138. Since the acetic acid, acetic anhydride and ethylene glycol diacetate are recycled in the process, the selectivity of separation of these fractions is generally not critical thereby reducing capital and energy costs.

Returning to line 138, the acetic anhydride is used in the process to react with 2-hydroxyethyl acetate and water and, optionally, converting the sulfuric acid catalyst to a less acidic acylated sulfate. Shown in the drawing are four options for the introduction of acetic anhydride:
(i) Line 140 introduces the acetic anhydride into the lower portion of distillation column 102 immediately above the bottoms section 108 or into a downcomer from that portion of the distillation column passing liquid phase to the bottoms section.
(ii) Line 142 introduces the acetic anhydride into bottoms section 108.
(iii) Line 144 introduces the acetic anhydride into line 120 carrying the bottoms liquid or vapor to reboiler 122.
(iv) Line 145 introduces the acetic anhydride into line 126 carrying the liquid phase ethylene glycol diacetate feed to cracking reactor 128.

The acetic anhydride can be introduced into one or more of these locations.

Returning to distillation column 132, a lights fraction is withdrawn as overhead via line 146. This lights fraction contains acetaldehyde which, if desired, could be recovered as a side product. It can be oxidized to acetic acid and returned to the process, or reacted with acetic anhydride to produce 1,1-diacetoxyethane for passing to cracking reactor 128 or another reactor for conversion to ethylene glycol diacetate. A liquid fraction containing vinyl acetate monomer is withdrawn from an upper region of column 146 and is passed via line 148 to reducing reactor 150. As water is generated by the reactions occurring in cracking reactor 128, the vinyl acetate monomer may be in an azeotrope with water. This liquid fraction contains butyraldehyde and other aldehydes that are difficult to separate by fractionation from vinyl acetate monomer. Reducing reactor 150 can contain a solid reducing agent or, as shown, a reducing agent, such as sodium borohydride, can be introduced via line 152 into the reactor. In the reactor, aldehydes are reduced to alcohols, e.g., butyraldehyde to butanol, to provide a substantially aldehyde-free product. The aldehyde-free product is passed to column 156 via line 154. Vinyl acetate monomer is withdrawn from the top of column 154 via line 158, and lines 160 and 162 withdraw butanol and propanol from column 156. Water can be removed from the vinyl acetate withdrawn via line 158 to provide a purified vinyl acetate product as is well known in the art.

Returning to distillation column 102, separation of water from acetic acid is conducted in non-reactive rectification section 106, with water being taken as overhead via line 164 and acetic acid dropping out of the vapor phase in section 106 as a downflowing liquid. As mentioned before, a portion of the monoethylene glycol feed can be provided into non-reactive rectification section 106 and serves to draw down the acetic acid, thereby reducing at least one of heat duty and distillation stages.

As shown, the optional use of an entrainer is depicted to facilitate the removal of water from acetic acid. For the purposes of this discussion, cyclohexane is used as the entrainer. Because of its low boiling point, it does not pass downwardly in the distillation column and dilute the reactants. As shown, the overhead from the distillation column is a water: cyclohexane azeotrope vapor, and the overhead passes via line 164 to liquid/liquid phase separator 166. An aqueous phase is withdrawn via line 168 and is passed to wastewater treatment. The organic phase, which contains cyclohexane and acetic acid, is withdrawn via line 170 and passed to a lower portion of non-reactive rectification section 106 or to the reactive distillation section, e.g., to a distillation stage immediately below the non-reactive rectification section.

Although the disclosure has been described with reference to preferred embodiments, persons skilled in the art will recognize that changes may be made in form and detail without departing from the spirit and scope of the disclosed apparatus, systems and methods.

1. A continuous process for making ethylene glycol diacetate from monoethylene glycol and acetic acid by reactive distillation comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having a reduced mass ratio of 2-hydroxyethyl acetate to ethylene glycol diacetate; and
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone.

2. The process of claim 1 wherein the crude ethylene glycol diacetate product contains at least 0.2 mass parts 2-hydroxyethyl acetate per 100 parts of ethylene glycol diacetate.

3. The process of claim 1 wherein the treated product contains less than about 0.1 parts by mass of 2-hydroxyethyl acetate per 100 parts of ethylene glycol diacetate.

4. The process of claim 1 wherein the crude ethylene glycol diacetate contains between about 0.5 to 5 parts by mass of 2-hydroxyethylacetate per 100 parts by mass of ethylene glycol diacetate when contacted with acetic anhydride in step (c) to make the treated product.

5. The process of claim 4 wherein the treated product contains less than 0.1 part by mass per 100 parts by mass of ethylene glycol diacetate.

6. The process of claim 5 wherein the treated product contains less than 50 parts per million of water.

7. The process of claim 4 wherein at least a portion of the treated product is subjected to cracking conditions to make vinyl acetate monomer and coproduce acetic anhydride, and at least a portion of the co-produced acetic anhydride is used as acetic anhydride in step (c).

8. The process of claim 4 in which the crude ethylene glycol diacetate product is contacted with acetic anhydride in a lower portion of the reactive distillation zone to provide the treated product.

9. The process of claim 4 in which the distillation zone comprises a reactive distillation zone and a bottoms section, and the crude ethylene glycol diacetate product is contacted with acetic anhydride in the bottoms section to provide the treated product.

10. The process of claim 4 in which the distillation zone comprises a reactive distillation zone and a bottoms section, and the crude ethylene glycol diacetate product is withdrawn from the bottoms section and is contacted with acetic anhydride to provide the treated product.

11. A continuous process for making vinyl acetate monomer from monoethylene glycol and acetic acid comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having a reduced mass ratio of 2-hydroxyethyl acetate to ethylene glycol diacetate;
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone;
e. continuously passing at least a portion of the treated product to a cracking zone maintained under conditions sufficient to convert at least a portion of the ethylene glycol diacetate to vinyl acetate monomer, wherein acetic acid in an amount of at least 0.5 mass parts per 100 mass parts of ethylene glycol acetate is also passed to the cracking zone, to provide a cracker effluent comprising vinyl acetate monomer and acetic anhydride;
f. continuously withdrawing cracker effluent from the cracking zone and selectively separating at least a portion of the acetic anhydride from the cracker effluent; and
g. passing at least a portion of the separated acetic anhydride to step (c).

12. The process of claim 11 wherein the cracking conditions comprise thermal cracking temperature of between about 490° C. and 550° C.

13. The process of claim 12 wherein the conversion of the ethylene glycol diacetate is between about 15 and 25 percent.

14. A continuous process for making vinyl acetate monomer from monoethylene glycol and acetic acid comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously contacting the crude ethylene glycol diacetate product with acetic anhydride in an amount and under conditions to provide a treated product having a reduced mass ratio of 2-hydroxyethyl acetate to ethylene glycol diacetate;
d. continuously withdrawing from the reactive distillation zone at least one of (i) the crude ethylene glycol diacetate product if step (c) is conducted outside the reactive distillation zone and (ii) treated product if step (c) is conducted inside the reactive distillation zone;
e. continuously passing at least a portion of the treated product to a cracking zone maintained under conditions sufficient to convert at least a portion of the ethylene glycol diacetate to vinyl acetate monomer, wherein acetic acid in an amount of at least 0.5 mass parts per 100 mass parts of ethylene glycol acetate is also passed to the cracking zone, to provide a cracker effluent comprising vinyl acetate monomer, acetaldehyde and acetic anhydride;
f. continuously withdrawing cracker effluent from the cracking zone and selectively separating at least a portion of the acetic anhydride from the cracker effluent and at least a portion of the acetaldehyde from the cracker effluent; and
g. contacting at least a portion of the separated acetic anhydride with at least a portion of the separated acetaldehyde under reaction conditions to produce a reaction product comprising 1,1-diacetoxyethane.

15. The process of claim 14 wherein at least a portion of the reaction product is passed to step (e) and at least a portion of the 1,1-diacetoxyethane is converted to vinyl acetate.

16. The process of claim 15 wherein acetic anhydride and acetaldehyde are admixed with the treated product passing to the cracking zone and 1,1-diacetoxyethane is produced in situ.

17. The process of claim 14 wherein the reaction conditions of step (e) comprise elevated temperature.

18. A continuous process for making ethylene glycol diacetate from monoethylene glycol and acetic acid by reactive distillation comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and to provide a crude ethylene glycol diacetate product containing ethylene glycol diacetate and 2-hydroxyethyl acetate;
b. continuously withdrawing the overhead containing water from the reactive distillation zone;
c. continuously passing the overhead from the reactive distillation zone to a non-reactive rectification zone, providing in the non-reactive rectification zone an azeotroping component having a boiling point less than that of acetic acid at the conditions in the non-reactive rectification zone, said azeotroping agent providing an azeotrope with water that has a boiling point less than that of water at the conditions in the non-reactive rectification zone, withdrawing a vapor phase overhead from the non-reactive rectification zone containing water and azeotroping agent having a reduced mole ratio of acetic acid to water than that of the overhead of step (b), and passing acetic acid downwardly in the non-reactive rectification zone to the reactive distillation zone; and
d. continuously withdrawing from the reactive distillation zone the crude ethylene glycol diacetate product.

19. The process of claim 18 wherein the azeotroping agent is an organic azeotroping agent, and the vapor phase overhead from the reactive distillation zone is condensed to provide an aqueous phase and an organic phase, and recycling at least a portion of the organic phase to step (c).

20. The process of claim 19 wherein the azeotroping agent is cyclohexane.
